(19) United States
(12) Patent Application Publication
(10) Pub. No.: US 2024/0139123 A1
(43) Pub. Date: May 2, 2024

(54) COMPOSITIONS AND METHODS FOR IMPROVING BRAIN FUNCTION

(71) Applicant: Health Via Modern Nutrition Inc., San Francisco, CA (US)

(72) Inventors: Geoffrey Hubert Woo, San Francisco, CA (US); Latt Shahril Mansor, San Francisco, CA (US); Christine Ensley, San Francisco, CA (US)

(21) Appl. No.: 18/549,148

(22) PCT Filed: Mar. 4, 2022
(86) PCT No.: PCT/US22/18935

§ 371 Date: Sep. 5, 2023

Related U.S. Application Data
(60) Provisional application No. 63/157,120, filed on Mar. 5, 2021.

(51) Int. Cl. A61K 31/047; A61K 9/00; A61K 9/16; A61K 9/50; A61K 31/00; A61K 36/185; A61K 47/12; A61K 47/46; A61P 25/22; A61P 25/28

(52) U.S. Cl. CPC A61K 31/047; A61K 9/0053; A61K 9/1652; A61K 9/1664; A61K 9/5052; A61K 9/5063; A61K 31/658; A61K 36/185; A61K 47/12; A61K 47/46; A61P 25/22; A61P 25/28

(57) ABSTRACT

Brain function may be improved by administering a therapeutically effective amount of 1,3-butanediol, selected from R-1,3-butanediol, S-1,3-butanediol, and racemic 1,3-butanediol, in combination with a therapeutically effective amount of cannabidiol (CBD). The disclosed compositions include synergistically effective amounts of 1,3-butanediol in combination with a synergistically effective amount of cannabidiol (CBD). The compositions may be administered to a subject for treating anxiety, concussion, traumatic brain injury (TBI), and/or cognitive impairment.

### RELATED APPLICATIONS

This application claims priority to U.S. Provisional Application Ser. No. 63/157,120 filed Mar. 5, 2021, and to U.S. Utility patent application Ser. No. 17/686,220 filed Mar. 3, 2022, each of which are incorporated herein by reference, in their entirety.

### FIELD OF INVENTION

The present invention relates to compositions and methods for improving brain function. The invention relates to compositions comprising therapeutically effective amounts of 1,3-butanediol, present as R-1,3-butanediol, S-1,3-butanediol, or racemic 1,3-butanediol, and cannabidiol (CBD). One or more disclosed compositions may be used to improve brain function, including, but not limited to, treating anxiety, concussion, traumatic brain injury (TBI), and/or cognitive impairment.

### BACKGROUND OF THE INVENTION

The present disclosure relates to compositions and methods for improving brain function in general. More particularly, the disclosure relates to compositions and methods for treating anxiety, concussion, traumatic brain injury (TBI), and/or cognitive impairment.

Anxiety refers to feelings of apprehension, unease, or fear. Physical symptoms of anxiety may include increased heart rate, sweating, rapid breathing, or trembling. Occasional anxiety is a normal part of life, but people with anxiety disorders frequently have intense, excessive and persistent worry and fear about everyday situations. Common anxiety disorders include generalized anxiety disorder, social anxiety disorder, separation anxiety disorder, and specific phobias.

Traumatic brain injury usually results from a violent blow or jolt to the head or body. An object that goes through brain tissue can also cause traumatic brain injury. Concussion is a type of traumatic brain injury that affects brain function. Effects can include headaches and problems with concentration, memory, balance and coordination.

Cognition refers to a range of mental processes relating to the acquisition, storage, manipulation, and retrieval of information. It underpins many daily activities. Cognitive functions, refer to various mental abilities, including learning, thinking, reasoning, remembering, problem solving, decision making, and attention. Improving and preserving cognitive functions are desirable at all ages.

CBD stands for cannabidiol. It is one of the two best-known active compounds derived from cannabis (marijuana). The other is tetrahydrocannabinol, or THC, which is the psychoactive substance that that produces the “high” from marijuana. CBD is not psychoactive and does not cause a “high.” While CBD is an essential component of medical marijuana, it is derived directly from the hemp plant, which is a cousin of the marijuana plant. Hemp contains 0.3% or less of THC. CBD has been shown to be effective at treating certain seizure disorders in children. There is moderate evidence that CBD can treat anxiety, pain, and certain sleep disorders. Current research is studying CBD as a neuroprotectant and brain treatment for concussion and brain injury.

Ketone bodies are the most energy-efficient fuel and yield more ATP per mole of substrate than pyruvate. Some recent studies have investigated ketones affecting cognitive and physical performance. Andrew J. Murray et al., “Novel ketone diet enhances physical and cognitive performance,” The FASEB Journal, Vol. 30, Issue 12, 2016, reported rats fed a diet supplemented with (R)-3-hydroxyburyl (R)-3-hydroxybutyrate as 30% of calories showed improved cognitive and physical performance. Mary T. Newport et al., “A new way to produce hyperketonemia: Use of ketone ester in case of Alzheimer's disease,” Alzheimer's & Dementia, Vol. 11, Issue 1, pp. 99-103, 2015, reported a 20-month oral administration of a ketone monoester to a patient with Alzheimer's disease dementia. The patient improved markedly in mood, affect, self-care, and cognitive and daily activity performance. The cognitive performance tracked plasma beta-hydroxybutyrate concentrations, with noticeable improvements in conversation and interaction at the higher levels, compared with predose levels. Mark Evans, et al., “Intermittent Running and Cognitive Performance after Ketone Ester Ingestion,” Medicine & Science in Sports & Exercise, November 2018, reported a 750 mg/kg ketone ester supplement attenuated the decline in executive function after exhausting exercise, suggesting a cognitive benefit after ketone ester ingestion. The ketone esters evaluated were R,S-1,3-butanediol acetoacetate ketone diester (KDE) and R-3-hydroxybutyl R-3-hydroxybutyrate ketone monoester (KME). It was reported that KDE would likely impair performance in high-intensity sports that demand a high rate of ATP provision from carbohydrate sources. Hunter S. Waldman et al., “Exogenous ketone salts do not improve cognitive responses after a high-intensity exercise protocol in healthy college-aged males,” Applied Physiology, Nutrition, and Metabolism, 16 Feb. 2018, examined the effects of a dl-β-hydroxybutyrate containing beverage on cognitive and performance measures during repeated Wingates (sprint interval training involving the repetition of “all out” 30 second efforts). No performance improvement was observed.

It would be an advancement in the art to provide novel and synergistic compositions and methods for improving brain function. It would also be an advancement in the art to provide novel and synergistic compositions and methods for treating subjects experiencing anxiety, concussion, traumatic brain injury (TBI), and/or cognitive impairment.

### BRIEF SUMMARY OF THE INVENTION

The present disclosure relates generally to compositions and methods for improving brain function. More specifically, the disclosed compositions may be used for treatment of anxiety, concussion, traumatic brain injury (TBI), and/or cognitive impairment. The disclosed compositions include a therapeutically effective amount of 1,3-butanediol in combination with a therapeutically effective amount of cannabidiol (CBD).

1,3-butanediol has the formula HOCH₂CH₂CH(OH)CH₃. It is a chiral diol. As used herein, “1,3-butanediol” includes R-1,3-butanediol, S-1,3-butanediol, or racemic 1,3-butanediol. R-1,3-butanediol is presently preferred.

The disclosed compositions may include CBD in any therapeutically effective form. The disclosed compositions may include CBD in an isolated and purified chemical form. The disclosed compositions may include CBD in naturally occurring botanical forms. The disclosed compositions may include CBD in a THC-free hemp extract. The disclosed compositions may include CBD in a hemp extract containing no more than 0.3% THC on a dry-weight basis.

The disclosed compositions include a synergistically effective amount of 1,3-butanediol, in the form of R-1,3-butanediol, S-1,3-butanediol, or racemic 1,3-butanediol, with R-1,3-butanediol being presently preferred, in combination with a synergistically effective amount of CBD. The disclosed compositions may be administered to a subject in a method of improving brain function. The disclosed compositions may be administered to a subject in a method for the treatment of anxiety, concussion, traumatic brain injury (TBI), and/or cognitive impairment.

Various embodiments are described herein. It will be understood that the embodiments listed below may be combined not only as listed below, but in other suitable combinations in accordance with the scope of the invention.

One or more disclosed compositions for improving brain function include therapeutically effective amounts of R-1,3-butanediol, S-1,3-butanediol or racemic 1,3-butanediol (preferably R-1,3-butanediol) and CBD. One or more disclosed compositions for the treatment of anxiety, concussion, traumatic brain injury (TBI), and/or cognitive impairment include therapeutically effective amounts of R-1,3-butanediol, S-1,3-butanediol or racemic 1,3-butanediol (preferably R-1,3-butanediol) and CBD.

The therapeutically effective amount of R-1,3-butanediol, S-1,3-butanediol or racemic 1,3-butanediol (preferably R-1,3-butanediol) in the compositions may range from about 1 mg to about 100 g, e.g., about 0.001 g to about 100 g, e.g., 0.001, 0.005, 0.01, 0.05, 0.1, 0.5, 1, 5, 10, 15, 20, 25, 30, 35, 40, 45, 50, 55, 60, 65, 70, 75, 80, 85, 90, 95, or 100 g, where any of the stated values can form an upper or lower endpoint of a range. In one or more embodiments, the therapeutically effective amount of R-1,3-butanediol, S-1,3-butanediol or racemic 1,3-butanediol (preferably R-1,3-butanediol) may range from about 5 g to about 35 g.

The therapeutically effective amount of CBD in the compositions may range from about 1 mg to about 100 mg, e.g., 1, 5, 10, 15, 20, 25, 30, 35, 40, 45, 50, 55, 60, 65, 70, 75, 80, 85, 90, 95, or 100 mg, where any of the stated values can form an upper or lower endpoint of a range. In one or more embodiments, the therapeutically effective amount of CBD may range from about 5 mg to about 50 mg.

One or more disclosed compositions may have a ratio (w/w) of CBD to R-1,3-butanediol, S-1,3-butanediol or racemic 1,3-butanediol (preferably R-1,3-butanediol) within a range from about 1:100 to about 1:7000. A presently preferred ratio (w/w) of CBD to R-1,3-butanediol, S-1,3-butanediol or racemic 1,3-butanediol (preferably R-1,3-butanediol) is about 1:200 to 1:400.

The disclosed invention includes one or more methods of improving brain function in a subject. The disclosed invention includes one or more methods for treating anxiety in a subject. The disclosed invention includes one or more methods for treating concussion in a subject. The disclosed invention includes one or more methods for treating traumatic brain injury (TBI) in a subject. The disclosed invention includes one or more methods for treating cognitive impairment in a subject.

The disclosed methods include administering to the subject any one of the disclosed compositions containing therapeutically effective amounts of R-1,3-butanediol, S-1,3-butanediol or racemic 1,3-butanediol (preferably R-1,3-butanediol) and CBD.

In one or more of the disclosed methods, the composition is administered when improved brain function is desired. In one or more of the disclosed methods, the composition is administered when treatment or prophylaxis of anxiety is desired. In one or more of the disclosed methods, the composition is administered when treatment or prophylaxis of brain injury in general is desired. In one or more of the disclosed methods, the composition is administered when treatment or prophylaxis of concussion or TBI is desired. In one or more of the disclosed methods, the composition is administered when treatment or prophylaxis of cognitive impairment is desired.

Without being bound by theory, ketones in the body provide a readily available substrate for brain metabolism in addition to glucose that can mitigate the damage from TBI. This is due to the observation that post-TBI, brain glucose metabolism is decreased, and this contributes to the damage in the neurons following TBI. It is presently believed that ensuring the brain has another form of substrate for energy may provide some mitigative properties as prophylaxis. In addition, the anti-inflammatory effects of CBD may also help as prophylaxis as much as treatment.

One or more disclosed compositions for improving brain function are synergistic compositions that include synergistically effective amounts of R-1,3-butanediol, S-1,3-butanediol or racemic 1,3-butanediol (preferably R-1,3-butanediol) and CBD. One or more disclosed compositions for the treatment of anxiety, concussion, traumatic brain injury (TBI), and/or cognitive impairment are synergistic compositions that include synergistically effective amounts of R-1,3-butanediol, S-1,3-butanediol or racemic 1,3-butanediol (preferably R-1,3-butanediol) and CBD.

The synergistically effective amount of R-1,3-butanediol, S-1,3-butanediol or racemic 1,3-butanediol (preferably R-1,3-butanediol) in the synergistic compositions may range from about 1 mg to about 100 g, e.g., about 0.001 g to about 100 g, e.g., 0.001, 0.005, 0.01, 0.05, 0.1, 0.5, 1, 5, 10, 15, 20, 25, 30, 35, 40, 45, 50, 55, 60, 65, 70, 75, 80, 85, 90, 95, or 100 g, where any of the stated values can form an upper or lower endpoint of a range. In one or more embodiments, the synergistic effective amount of R-1,3-butanediol, S-1,3-butanediol or racemic 1,3-butanediol (preferably R-1,3-butanediol) may range from about 10 g to about 35 g.

The synergistically effective amount of CBD in the synergistic compositions may range from about 1 mg to about 100 mg, e.g., 1, 5, 10, 15, 20, 25, 30, 35, 40, 45, 50, 55, 60, 65, 70, 75, 80, 85, 90, 95, or 100 mg, where any of the stated values can form an upper or lower endpoint of a range. In one or more embodiments, the synergistically effective amount of CBD may range from about 5 mg to about 50 mg.

One or more disclosed synergistic compositions may have a ratio (w/w) of CBD to R-1,3-butanediol, S-1,3-butanediol or racemic 1,3-butanediol (preferably R-1,3-butanediol) within a range from about 1:100 to about 1:7000. A presently preferred ratio (w/w) of CBD to R-1,3-butanediol, S-1,3-butanediol or racemic 1,3-butanediol (preferably R-1,3-butanediol) is about 1:200 to 1:400.

The disclosed invention includes one or more methods of improving brain function in a subject. The disclosed invention includes one or more methods for treating anxiety in a subject. The disclosed invention includes one or more methods for treating concussion in a subject. The disclosed invention includes one or more methods for treating traumatic brain injury (TBI) in a subject. The disclosed invention includes one or more methods for treating cognitive impairment in a subject.

The disclosed methods include administering to the subject any one of the disclosed synergistic compositions containing synergistically effective amounts of R-1,3-butanediol, S-1,3-butanediol or racemic 1,3-butanediol (preferably R-1,3-butanediol) and CBD.

In one or more of the disclosed methods, the synergistic composition is administered when improved brain function is desired. In one or more of the disclosed methods, the synergistic composition is administered when treatment or prophylaxis of anxiety is desired. In one or more of the disclosed methods, the synergistic composition is administered when treatment or prophylaxis of brain injury in general is desired. In one or more of the disclosed methods, the synergistic composition is administered when treatment or prophylaxis of concussion or TBI is desired. In one or more of the disclosed methods, the synergistic composition is administered when treatment or prophylaxis of cognitive impairment is desired.

The disclosed compositions may be provided in any oral consumable form. It is also within the scope of the disclosed invention to configure the disclosed compositions into formulations suitable for parenteral (including subcutaneous, intradermal, intramuscular, and intravenous) and rectal administration. In some embodiments, the disclosed compositions are in the form of a tablet, capsule, or pill suitable for oral administration. In some embodiments, the disclosed compositions are in the form of a solid foodstuff such as a snack food, nutritional bar, or protein bar. In some embodiments, the disclosed compositions are in liquid formulations (e.g., water, carbonated beverages, soft drinks, milk, juice, tea, fermented beverages) suitable for oral administration. In some embodiments, the disclosed compositions are in the form of powders that can be used to prepare drink mixes or can be added as a supplement to other food or drink products. One or more pharmaceutically acceptable carriers may be provided. In various embodiments, the compositions are formulated for oral administration, including immediate release, extended release, and sustained release formulations.

It is to be understood that both the foregoing general description and the following detailed description are examples and explanatory and are not restrictive of the invention, as claimed. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### DETAILED DESCRIPTION OF THE INVENTION

The present invention relates to compositions and methods for improving brain function in general. More particularly, the invention relates to compositions and methods for treating anxiety, concussion, traumatic brain injury (TBI), and/or cognitive impairment.

The invention relates to compositions comprising therapeutically effective amounts of R-1,3-butanediol, S-1,3-butanediol, or racemic 1,3-butanediol (preferably R-1,3-butanediol) and cannabidiol (CBD).

The terminology used herein is for the purpose of describing particular embodiments only and is not intended to be limiting. In this specification and in the claims that follow, reference will be made to a number of terms, which shall be defined to have the definitions set forth below. Unless otherwise defined, all technical and scientific terms used herein have the same meaning as commonly understood by one of ordinary skill in the art to which this invention belongs. The singular forms also include the plural unless the context clearly dictates otherwise. Thus, the singular forms “a,” “an” and “the” include plural referents unless the context clearly dictates otherwise.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. Additionally, while the following description refers to several embodiments and examples of the various components and processes of the described invention, all of the described embodiments and examples are to be considered, in all respects, as illustrative only and not as being limiting in any manner.

As used herein, the expression [A], [B], [C], “and/or” [D] means that one or more of the cases connected by the expression “and/or” may occur individually or in combination. Thus, the expression means [A] or [B] or [C] or [D] may occur individually, or combinations of any two or more cases may occur, such as [A] and [B], [A] and [C], [B] and [C], [A], [C], and [D], etc.

As used herein, unless explicitly stated otherwise or clearly implied otherwise, the term “about” refers to a range of values plus or minus 10 percent (“±10%”), e.g. about 1.0 encompasses values from 0.9 to 1.1.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the range or the characteristics being described. Furthermore, the described features, structures, characteristics, processes, or methods of the invention may be combined in any suitable manner in one or more embodiments.

“Active agent” and “therapeutic agent” means a compound that exerts a positive therapeutic effect on the health and well-being of a subject. Active agent can refer not only to a single active agent but also to a combination of two or more different active agents.

As used herein, the term “beverage” can relate to consumable liquids including but not limited to water, carbonated water, naturally and/or artificially flavored waters, soft drinks, teas, juices, milk, extractions, fermented beverages, alcohol-containing beverages, and other known consumable liquids. In some embodiments, the beverage is provided in a single-dose container for consumer use. Alternatively, the beverage is provided in a multi-dose container.

“Sustained release” and “extended release” means a composition containing an active agent that provides for gradual release of the active agent over an extended period of time, and typically, although not necessarily, results in substantially constant blood levels of an active agent over an extended time period.

“Dosage form” means any form of a composition for administration to a subject (typically a human seeking a therapeutic or synergistic effect). “Dose” refers to an amount of active agent. A single tablet or capsule is a unit dosage form. Multiple unit dosage forms can be administered to provide a therapeutically effective dose. A dosage form can include a combination of dosage forms.

“Effective amount,” “therapeutically effective amount,” and “synergistically effective amount” refer to a nontoxic but sufficient amount of an active agent to achieve a desired therapeutic or synergistic effect.

The term “composition” refers to a composition that is suitable for administration to a subject. In general a “composition” is sterile, and preferably free of contaminants that are capable of eliciting an undesirable response within the subject.

The term “natural flavor” or “natural flavoring” means the essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, whose significant function in food and supplement is flavoring rather than nutritional.

Percentages and ratios used herein, unless otherwise indicated, are by weight.

“Treating” and “treat” includes the administration to a subject one or more doses of an active agent to affect the condition by improving or altering it or to obtain a desired therapeutic or synergistic effect.

Certain compositions of the disclosed invention comprise a therapeutically effective amount of R-1,3-butanediol, S-1,3-butanediol, or racemic 1,3-butanediol (preferably R-1,3-butanediol) in combination with a therapeutically effective amount of cannabidiol (CBD). The compositions may also comprise one or more pharmaceutically acceptable (approved by a state or federal regulatory agency for use in humans, or is listed in the U.S. Pharmacopia, the European Pharmacopia) excipients or carriers. The terms “excipient” or “carrier” as used herein broadly refers to a biologically inactive substance used in combination with the active agents of the formulation. An excipient can be used, for example, as a solubilizing agent, a stabilizing agent, a diluent, an inert carrier, a preservative, a binder, a disintegrant, a coating agent, a flavoring agent, or a coloring agent. Preferably, at least one excipient is chosen to provide one or more beneficial physical properties to the formulation, such as increased stability and/or solubility to the therapeutic agents.

Non-limiting examples of suitable excipients for liquid or beverage formulations include flavoring agents, sweeteners, including nutritive and non-nutritive sweeteners, acidifiers such as citric, malic acid, tartaric acid, and phosphoric acid, and emulsifiers such as hydrocolloids like xanthan, gum acacia and gum acacia, modified starches, pectin, carrageenan, casein, and inulin. Non-limiting examples of suitable excipients for solid formulations include flow agents such as silicon dioxide, magnesium stearate, and stearic acid, binders such as guar gum, xanthan gum, and acacia gum, carriers such as naturally occurring complex carbohydrates, acidifiers such as naturally-occurring acids including citric acid, malic acid, tartaric acid, and aspartic acid.

Solutions and suspensions used for the delivery can include the following components: a sterile diluent such as water for injection, saline solution, fixed oils, polyethylene glycols, glycerine, polylene glycol, polysorbate, tocopherol polyethylene glycol succinate (TPGS), or other synthetic solvents; antibacterial agents such as benzyl alcohol or methyl parabens; antioxidants such as ascorbic acid or sodium bisulfite; chelating agents such as ethylenediamineteraacetic acid; buffers such as acetates, citrates or phosphates, and agents for the adjustment of tonicity such as sodium chloride or dextrose. The pH can be adjusted with acids or bases, such as hydrochloric acid or sodium hydroxide.

In some embodiments, the compositions for improving brain function and for treating anxiety, concussion, traumatic brain injury (TBI), and/or cognitive impairment of the presently disclosed invention are prepared in a solid form such as a powder, tablet, pill or capsule for oral administration. In alternative embodiments, liquid formulations for oral administration may take such forms as water, carbonated beverages, soft drinks, fermented beverage suspensions, solutions and emulsions, and may contain formulatory agents such as suspending, stabilizing and/or dispersing agents. In some embodiments, the disclosed compositions may be in the form of powders that can be used to prepare drink mixes or can be added as a supplement to other food or drink products.

The dosage forms, e.g., an oral dosage form, may provide for rapid release or provide for extended release or sustained release, i.e., gradual, release of the R-1,3-butanediol, S-1,3-butanediol or racemic 1,3-butanediol (preferably R-1,3-butanediol) and the CBD from the dosage form to the subject's body over an extended time period, typically providing for a substantially constant blood level of the therapeutic agents over a time period in the range of about 4 to about 24 hours, typically in the range of about 4 to about 12 hours, or of about 6 to about 10 hours.

In some embodiments, it may be especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of a first or second therapeutic agent, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are another oral dosage form of the present invention, wherein the R-1,3-butanediol, S-1,3-butanediol, or racemic 1,3-butanediol (preferably R-1,3-butanediol) and the CBD are encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like.

Oral dosage forms, whether tablets, capsules, caplets, or particulates, if desired, may be formulated so as to provide for extended or controlled release of the R-1,3-butanediol, S-1,3-butanediol, or racemic 1,3-butanediol (preferably R-1,3-butanediol) and the CBD.

Generally, as will be appreciated by those of ordinary skill in the art, extended release and sustained release dosage forms are formulated by dispersing at least one of the two or more active therapeutic agents within a matrix of a gradually hydrolyzable material such as a hydrophilic polymer, or by coating a solid, active agent-containing dosage form with such a material. Hydrophilic polymers useful for providing an extended release or a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Other features and advantages of the present invention are apparent from the different examples that follow. The examples below illustrate different aspects and embodiments of the present invention and how to make and practice them. The examples do not limit the claimed invention. Although methods and materials similar or equivalent to those described herein can be used in the practice of the present invention, suitable methods and materials are described below. Based on the present disclosure the skilled artisan can identify and employ other components and methodology useful for practicing the present invention.

### Examples 1-6: Therapeutically Effective Composition of R-1,3-Butanediol and CBD

Examples 1-6 describe a process for preparing several therapeutically effective compositions of R-1,3-butanediol and CBD suitable for oral administration. The ingredients are mixed in water in the amounts set forth in Table 1. Citric acid (anhydrous) is used as the pH control. Thereafter, the composition is processed by heat until the liquid temperature reaches 165° F., bottled into a consumer-acceptable form, and cooled to a temperature of no more than 95° F.

TABLE 1

| | Example | | | | | |
|---|---|---|---|---|---|---|
| | 1 | 2 | 3 | 4 | 5 | 6 |
| Water (mL) | 280 | 500 | 500 | 375 | 475 | 250 |
| R-1,3-Butanediol (g) | 10 | 5 | 20 | 10 | 25 | 10 |
| CBD (mg) | 10 | 20 | 20 | 25 | 25 | 30 |
| Natural Flavor (mg) | 300 | 500 | 500 | 400 | 450 | 300 |
| pH Control (mg) | 800 | 1200 | 1200 | 1000 | 1100 | 800 |

### Example 7: Therapeutically Effective Composition of R-1,3-Butanediol and CBD

This example describes a process for preparing a therapeutically effective composition of R-1,3-butanediol and CBD suitable for oral administration. The composition is prepared by mixing 475 mL water and 15 g R-1,3-butanediol. CBD in the form of Hemp Extract containing therein 25 mg CBD, is mixed with the composition until dissolved. 60 mg of Monk Fruit Extract is added to the composition and mixed until dissolved. 1500 mg lemon juice is added to the composition. Thereafter, the composition is processed by heat until the liquid temperature reaches 165° F., bottled into a consumer-acceptable form, and cooled to a temperature of no more than 95° F.

### Example 8: Therapeutically Effective Composition of R-1,3-Butanediol and CBD, in Powder Mix Form

This example describes a process for preparing a therapeutically effective composition of R-1,3-butanediol and CBD in powder mix form, for oral administration after mixing with liquid by the consumer. The composition may be prepared by blending ingredients in powder form. Powder forms of individual ingredients or combinations of ingredients may be obtained by spray drying liquid forms of individual ingredients or combinations of ingredients.

In an example, the composition is prepared by spray drying a mixture of 50 g R-1,3-butanediol and 100 g gum acacia to form a powder. 50 mg of a powdered CBD extractare blended with the composition until evenly distributed. 5 g of a powder FDA-compliant natural flavor and 5 g coconut cream powder are blended with the composition until evenly distributed. Thereafter, the composition is bottled into a consumer-acceptable form. In use, about 20 g of the resulting composition is mixed in 8 oz. water and administered to a subject.

### Example 9: Therapeutically Effective Composition of R-1,3-Butanediol and CBD

This example describes a process for preparing a therapeutically effective composition of R-1,3-butanediol and CBD suitable for oral administration. The composition is prepared by mixing 10 g R-1,3-butanediol and CBD in the form of Hemp Extract containing therein 25 mg CBD. Thereafter, the blend is encapsulated into gelatin softgel capsules and coated with lemon extract.

### Example 10: Method for Improving Brain Function

Subjects are administered a composition comprising 20-30 g R-1,3-butanediol and 25-30 mg CBD. Approximately 30-60 minutes after consumption, the subjects are asked to go about their daily tasks. The subjects are asked to complete a questionnaire and rate their subjective feeling on cognitive performance compared to CBD alone. The questionnaire asked the subjects to rate on a scale of 1-10, with 1 being “Less than normal” and 10 being “Higher than normal” (1) levels of focus and mental energy.

### Example 11: Method for Treatment of Anxiety

Subjects are administered a composition comprising 20-30 g R-1,3-butanediol and 25-30 mg CBD. Approximately 60-90 minutes after consumption, the subjects are asked to complete a questionnaire and rate their subjective feeling on ‘mental state’. The questionnaire asks the subjects to rate on a scale of 1-10, with 1 being “Anxious” and 10 being “Very relaxed” on the relaxation and anxiolytic effect.

### Example 12: Method for Treatment of Concussion and Traumatic Brain Injury

The inclusion criteria of the subjects for this study should be 1) have a high risk of brain injury and 2) have had previous brain injury. The example population are football players or military personnel. Subjects are administered a composition comprising 20-30 g R-1,3-butanediol and 25-30 mg CBD prior to a training session, a match or military course where they are exposed to the risk of brain injury. Subjects are monitored closely and if they experience any form of injury, they have to complete a questionnaire on a scale 1-10, with 1 being “Mild” and 10 being “Severe”, on the subject feel on the damage of the injury compared to the previous injury they have experienced. Over the next 7-14 days, these injured subjects are administered a composition comprising 20-30 g R-1,3-butanediol and 25-30 mg CBD daily and they should also complete a questionnaire on their recovery rate daily on a scale 1-10 with 1 being “Slightly Better” and 10 being “Much better” compared to the first day of injury.

### Example 13: Method for Treatment of Cognitive Impairment

Subjects with mild cognitive impairment are chosen for this study. Subjects are tested using standardized cognitive/memory tests to establish baseline measurements. Subjects are administered a composition comprising 20-30 g R-1,3-butanediol and 25-30 mg CBD daily, for 14 days. Subjects are then tested again using the same standardized cognitive and memory tests on days 3, 7, 10 and 14. Results will be compared to baseline measurements to investigate the effectiveness of said composition in improving cognitive performance in the mild cognitive impaired individuals.

Synergistic Activity of R-1,3-Butanediol and CBD

Without being bound by theory, it is presently believed a synergistic effect is obtained by the co-administration of a synergistically effective amount of 1,3-butanediol, present as R-1,3-butanediol, S-1,3-butanediol, or racemic 1,3-butanediol (preferably R-1,3-butanediol), in combination with a synergistically effective amount of CBD.

It is presently believed synergistic effects between R-1,3-butanediol, S-1,3-butanediol, or racemic 1,3-butanediol (preferably R-1,3-butanediol) and CBD are obtained in brain function improvement, including prophylaxis and treatment for anxiety, concussion, traumatic brain injury (TBI), and/or cognitive impairment.

Anxiety

CBD exhibited an anti-anxiety and antidepressant effects in animal models (de Mello Schier A R, de Oliveira Ribeiro N P, Coutinho D S, Machado S, Arias-Carión O, Crippa J A, Zulardi A W, Nardi A E, Silva A C. Antidepressant-like and anxiolytic-like effects of cannabidiol: a chemical compound of Cannabis sativa. CNS Neurol Disord Drug Targets. 2014:13(6):953-60. doi: 10.2174/1871527313666140612114838. PMID: 24923339). CBD was also shown to have anxiolytic effect via treatments in teenagers with social anxiety disorders (Masataka N (2019) Anxiolytic Effects of Repeated Cannabidiol Treatment in Teenagers With Social Anxiety Disorders. Front. Psychol. 10:2466. doi: 10.3389/fpsyg.2019.02466). Animal studies showed that ketones also exhibit anxiolytic effect via regulation of GABA (Sang Woo Kim et al., Ketone beta-hydroxybutyrate up-regulates BDNF expression through NF-κB as an adaptive response against ROS, which may improve neuronal bioenergetics and enhance neuroprotection, Neurology April 2017, 88 (16 Supplement) P3.090), activation of brain derived neurotrophic factor, as well as via adenosine A1 receptors (Kovács Z, D'Agostino D P and Ari C (2018) Anxiolytic Effect of Exogenous Ketone Supplementation is Abolished by Adenosine A1 Receptor Inhibition in Wistar Albino Glaxo/Rijswijk Rats. Front. Behav. Neurosci. 12:29. doi: 10.3389/fnbeh.2018.00029). In addition, CBD was also shown to affect adenosine A1 receptors (Gonca E, Darici F. The effect of cannabidiol on ischemia/reperfusion-induced ventricular arrhythmias: the role of adenosine A1 receptors. J Cardiovasc Pharmacol Ther. 2015 January; 20(1):76-83. doi: 10.1177/1074248414532013. Epub 2014 May 22. PMID: 24853683), suggesting that the synergy between ketones and CBD in providing anxiolytic effects may be additive compared to the effect of ketones or CBD alone.

Concussion and Traumatic Brain Injury.

In traumatic brain injury, altered NF-κB activation was shown in mice (Sullivan P G, Bruce-Keller A J, Rabchevsky A G, Christakos S, Clair D K, Mattson M P, Scheff S W. Exacerbation of damage and altered NF-kappaB activation in mice lacking tumor necrosis factor receptors after traumatic brain injury. J Neurosci. 1999 Aug. 1; 19(15):6248-56. doi: 10.1523/JNEUROSCI.19-15-06248.1999. PMID: 10414954; PMCID: PMC6782813). This is in line with another study (Nonaka M, Chen X H, Pierce J E, Leoni M J, McIntosh T K, Wolf J A, Smith D H. Prolonged activation of NF-kappaB following traumatic brain injury in rats. J Neurotrauma. 1999 November; 16(11):1023-34. doi: 10.1089/neu.1999.16.1023. PMID: 10595819) that showed prolonged NF-κB activation in regions undergoing persistent atrophy following TBI, suggesting the role of NF-κB activation in long-term inflammatory processes following TBI. In vitro studies showed CBD inhibits reactive oxygen species (ROS) production by mitochondria and NF-κB activation (Rajesh M, Mukhopadhyay P, Bátkai S, Haskó G, Liaudet L, Drel V R, Obrosova T G, Pacher P. Cannabidiol attenuates high glucose-induced endothelial cell inflammatory response and barrier disruption. Am J Physiol Heart Circ Physiol. 2007 July; 293(1):H610-9. doi: 10.1152/ajpheart.00236.2007. Epub 2007 Mar. 23. PMID: 17384130; PMCID: PMC2228254) and since NF-κB activation may play such a significant role in traumatic brain injury, CBD may confer protective properties to the injury itself as well as the long-term damage that follows. On top of that, CBD has shown to have strong anti-inflammatory properties in both animal (Sumariwalla P F, Gallily R, Tchilibon S, Fride E, Mechoulam R, Feldmann M. A novel synthetic, nonpsychoactive cannabinoid acid (HU-320) with antiinflammatory properties in murine collagen-induced arthritis. Arthritis Rheum. 2004 March; 50(3):985-98. doi: 10.1002/art.20050. PMID: 15022343) and in vitro studies (Rajesh M, Mukhopadhyay P. Bátkai S, Haskó G, Liaudet L. Drel V R, Obrosova I G, Pacher P. Cannabidiol attenuates high glucose-induced endothelial cell inflammatory response and barrier disruption. Ain J Physiol Heart Circ Physiol. 2007 July; 293(1):H610-9. doi: 10.1152/ajpheart.00236.2007. Epub 2007 Mar. 23. PMID: 17384130; PMCID: PMC2228254), which may be extremely beneficial for brain injuries. The post-brain injury period of glucose metabolic depression is accompanied by adenosine triphosphate decreases and free radical production (Mayumi L. Prins, Joyce H. Matsumoto, The collective therapeutic potential of cerebral ketone metabolism in traumatic brain injury, Journal of Lipid Research, Volume 55, Issue 12, 2014, Pages 2450-2457, ISSN 0022-2275, https://doi.org/10.1194/jlr.R046706). Given these events following a brain injury, it is essential to first, mediate the damage from oxidative damage and free radical production and second, provide the brain with the only natural alternative substrates to glucose, that is ketones, to mitigate the damage driven by the metabolic deficiencies in the brain (White H, Venkatesh B. Clinical review: ketones and brain injury. Crit Care. 2011 Apr. 6; 15(2):219. doi: 10.1186/cc10020. PMID: 21489321 PMCID: PMC3219:306). Another animal study showed the potential of ketones in treating head injuries in younger patients (M. L. Prins, L. S. Fujima, D. A. Hovda, Age-dependent reduction of cortical contusion volume by ketones after traumatic brain injury, J. Neuroscience research, Vol. 82, Issue 3, pp. 413-420, 2005, https://doi.org/10.1002/jnr.20633). We believe that ketones and CBD works synergistically as prophylaxis as well as treatment to brain injury by providing the advantage in terms of cell signaling pathways as well as substrate utilization and metabolism.

Cognitive Impairment

CBD was shown to improve cognitive impairment and reverse cortical transcriptional changes in Schizophrenia-like rat model (Kozela E, Krawczyk M, Kos T, Juknat A, Vogel Z, Popik P. Cannabidiol Improves Cognitive Impairment and Reverses Cortical Transcriptional Changes Induced by Ketamine, in Schizophrenia-Like Model in Rats. Mol Neurobiol. 2020 March; 57(3):1733-1747. doi: 10.1007/s12035-019-01831-2. Epub 2019 Dec. 11. PMID: 31823199). Another study demonstrated CBD's ability to prevent the development of a social recognition deficit in Alzheimer's disease transgenic mice (Cheng D, Spiro A S, Jenner A M, Garner B, Karl T. Long-term cannabidiol treatment prevents the development of social recognition memory deficits in Alzheimer's disease transgenic mice. J Alzheimers Dis. 2014; 42(4):1383-96. doi: 10.3233/JAD-140921. PMID: 25024347). This review article also detailed the potential usage of CBD for Alzheimer's disease (Watt C, Karl T. In vivo Evidence for Therapeutic Properties of Cannabidiol (CBD) for Alzheimer's Disease. Front Pharmacol. 2017 Feb. 3; 8:20, doi: 10.3389/fphar.2017.00020. PMID: 28217094; PMCID: PMC5289988). Ketones on the other hand has been proven to increase brain network stability in humans (Lilianne R. Mujica-Parodi, Anar Aingalan, Syed Fahad Sultan, Botond Antal, Xiaofei Sun, Steven Skiena, Andrew Lithen, Noor Adra, Eva-Maria Ratai, Corey Weistuch, Sindhuja Tirumalai Govindarajan, Helmut H. Strey, Ken A. Dill, Steven M. Stufflebeam, Richard L. Veech, Kieran Clarke, Diet modulates brain network stability, a biomarker for brain aging, in young adults, Proceedings of the National Academy of Sciences, March 2020, 117 (11) 6170-6177; DOI: 10.1073/pnas.1913042117). A 6-month randomized controlled trial showed cognitive improvement in the mild cognitive impaired individuals (Fortier, M, Castellano, C-A, St-Pierre, V, et al. A ketogenic drink improves cognition in mild cognitive impairment: Results of a 6-month RCT. Alzheimer's Dement. 2020; 1-10; https://doi.org/10.1002/alz.12206). We believe there are synergistic effects between ketone and CBD in improving cognitive decline and impairment, given the current data.

All publications, patents, patent applications, or other documents cited herein are hereby incorporated by reference in their entirety for all purposes to the same extent as if each individual publication, patent, patent application, or other document was individually indicated to be incorporated by reference for all purposes.

While this disclosure has been particularly shown and described with references to preferred embodiments thereof, it will be understood by those skilled in the art that various changes in form and details may be made therein without departing from the scope of the disclosure encompassed by the appended claims.

1. A composition comprising active agents consisting essentially of therapeutically effective amounts of 1,3-butanediol and Cannabidiol (CBD), wherein such composition is in the form of a tablet, capsule, or pill suitable for oral administration, a solid foodstuff such as a snack food, nutritional bar, or protein bar, a liquid formulation suitable for oral consumption or a powder that can be used to prepare drink mixes or can be added as a supplement to other food or drink products.

2. The composition of claim 1, wherein the 1,3-butanediol is selected from R-1,3-butanediol, S-1,3-butanediol, and racemic 1,3-butanediol.

3. The composition of claim 2, comprising from 1 mg to 100 g 1,3-butanediol, and from 1 mg to 100 mg CBD.

4. The composition of claim 2, comprising from 5 g to 35 g 1,3-butanediol and from 5 mg to 50 mg CBD.

5. The composition of claim 1, wherein said composition is prepared in a liquid formulation suitable for oral consumption.

6. The composition of claim 1, comprising a ratio (w/w) of CBD to 1,3-butanediol within a range from 1:100 to 1:7000.

7. The composition of claim 1, wherein the 1,3-butanediol is R-1,3-butanediol.

8. A composition comprising active agents consisting essentially of synergistically effective amounts of 1,3-butanediol and cannabidiol (CBD), wherein such composition is in the form of a tablet, capsule, or pill suitable for oral administration, a solid foodstuff such as a snack food, nutritional bar, or protein bar, a liquid formulation suitable for oral consumption or a powder that can be used to prepare drink mixes or can be added as a supplement to other food or drink products.

9. The composition of claim 8, wherein the 1,3-butanediol is selected from R-1,3-butanediol, S-1,3-butanediol, and racemic 1,3-butanediol.

10. The composition of claim 8, comprising a ratio (w/w) of CBD to 1,3-butanediol within a range from 1:100 to 1:7000.

11. The composition of claim 8, comprising from 1 mg to 100 g 1,3-butanediol and from 1 mg to 300 mg CBD.

12. The composition of claim 8, comprising from 1 g to 50 g 1,3-butanediol and from 1 mg to 150 mg CBD.

13. The composition of claim 8, wherein said composition is prepared in a liquid formulation suitable for oral consumption.

14. The composition of claim 8, wherein the 1,3-butanediol is R-1,3-butanediol.

15. A method for improving brain function in a subject, comprising administering to the subject a composition comprising 1,3-butanediol; and cannabidiol (CBD).

16. The method of claim 15, wherein the 1,3-butanediol is selected from R-1,3-butanediol, S-1,3-butanediol, and racemic 1,3-butanediol.

17. The method of claim 15, wherein the composition comprises 1,3-butanediol in a therapeutically effective amount from 1 mg to 100 g; and CBD in a therapeutically effective amount from 1 mg to 100 mg.

18. The method of claim 15, wherein the composition comprises 1,3-butanediol in a synergistically effective amount of from 1 mg to 100 g, and CBD in a synergistically effective amount of from 1 mg to 300 mg.

19. The method of claim 15, wherein said composition is prepared in a liquid formulation suitable for oral consumption.

20. The method of claim 15 wherein the 1,3-butanediol is R-1,3-butanediol.

21. The method of claim 15, wherein the brain function is selected from the group consisting of anxiety, concussion, traumatic brain injury, and cognitive impairment.

22. A method of improving brain function in a subject, the method comprising the step of administering to the subject a composition according to claim 1.

23. The method of claim 22, wherein the brain function is selected from the group consisting of anxiety, concussion, traumatic brain injury, and cognitive impairment.

24. A method of improving brain function in a subject, the method comprising the step of administering to the subject a synergistic composition according to claim 8.

25. The method of claim 24, wherein the brain function is selected from the group consisting of anxiety, concussion, traumatic brain injury, and cognitive impairment.
